(19) United States
(12) Patent Application Publication
(10) Pub. No.: US 2010/0179469 A1
(43) Pub. Date: Jul. 15, 2010

(54) Organic Light Emitting Diode Phototherapy Lighting System

(75) Inventors: Troy Hammond, Pittsburgh, PA (US); Sujit Naik, Pittsburgh, PA (US); Lisa Pattison, Morgantown, WV (US)

Correspondence Address: TROUTMAN SANDERS LLP;5200 BANK OF AMERICA PLAZA, 600 PEACHTREE STREET, N.E., SUITE 5200, ATLANTA, GA, 30308-2216, US

(73) Assignee: Plextronics, Inc., Pittsburgh, PA (US)

(21) Appl. No.: 12/652,359

(22) Filed: Jan. 5, 2010

Related U.S. Application Data
(60) Provisional application No. 61/142,403, filed on Jan. 5, 2009.
(60) Provisional application No. 61/167,352, filed on Apr. 7, 2009.

(51) Int. Cl. A61N 5/06

(52) U.S. Cl. 604/20; 607/88; 607/90; 607/91; 607/92

(57) ABSTRACT

A lighting system includes a plurality of organic light emitting diode (OLED) devices. By selecting the plurality of OLED devices, or by selectively controlling the plurality of OLED devices, the color characteristics of the lighting system can be tuned. The lifespan of the lighting system can be improved.

This application claims benefit, under 35 U.S.C. §119(e), from U.S. Provisional Application Ser. No. 61/142,403 filed on Jan. 1, 2009 and U.S. Provisional Application Ser. No. 61/167,352 filed on Apr. 7, 2009, the entire contents of which are hereby incorporated by reference.

### TECHNICAL FIELD OF THE INVENTION

The invention is directed to a phototherapy device. Embodiments of the invention are directed to phototherapy devices comprising at least one organic light emitting diode (“OLED”). OLED phototherapy devices are capable of providing emission spectrums targeted for treatment of a specific disease or disorder with variable intensity and lower heat output than conventional phototherapy devices.

### BACKGROUND

Certain illnesses may be treated with exposure to light. The treatment may include light exposure alone or in combination with photosensitive medications. Such treatments are generally referred to as phototherapy. Phototherapy has been used for the treatment of neonatal jaundice, acne, psoriasis, eczema, cancer, neuropathy, seasonal affective disorders, depression, bulimia, tremors associated with Parkinson's disease, ulcers, and circadian rhythm maintenance disorders as well as other illnesses, disorders, or diseases, for example. Phototherapy has also been used to facilitate wound healing, promote relaxation, improve fertility, stimulate hair growth and decrease the appearance of cellulite and wrinkles. Several phototherapy devices have been developed for treatment of such illnesses. The devices are typically designed to deliver specifically controlled light to the patient. Such devices may use fluorescent lamps, halogen lamps, or inorganic light emitting diodes as a direct spot light or in combination with fiber optics. Designs incorporating organic light emitting diodes are generally bandage embodiments that target a small area of affected tissue and are ambulatory devices.

The effectiveness of a phototherapy treatment typically depends on the intensity of the light, the surface area of exposure to the light, and the percentage of the light spectrum comprising wavelengths effective for treatment of the disease or disorder.

Organic light emitting diodes (OLEDs) comprise chemical compounds that produce light when the electrons in the chemical compound are transferred into their excited state when subject to an external power source. The compounds are generally classified as organic polymers or as small molecules. Accordingly, OLED devices may be referred to as polymer light emitting diode (PLED) devices or small molecule organic light emitting diode (SMOLED) devices depending on the composition of their active regions. Earlier OLEDs were typically based on relatively simple structures, where a thin layer of the electroluminescence (EL) conjugated polymer was enclosed between a pair of electrodes. When a voltage is applied to the electrodes, the positive (anode) and the negative (cathode) electrodes can provide injection of holes and electrons, respectively, into the EL polymer. In the EL polymer layer, electrons and holes move towards each other in the applied electrical field and form excitons, which are bound excited states that can relax down into the ground state radiatively by emitting a photon. This process can be referred to as electroluminescence.

OLEDs were first designed in the 1980's, see, e.g., C. W. Tang, S. A. Van Slyke, Organic electroluminescent diodes, Appl. Phys. Lett. 1987, 51, 913. More recent developments in OLED materials and applications are generally described in Kraft et al., Angew. Chem. Int. Ed., 1998, 37, 402-428, and Z., Li and H. Meng, Organic Light-Emitting Materials and Devices (Optical Science and Engineering Series), CRC Taylor & Francis (Sep. 12, 2006). All references cited in this application are hereby incorporated by reference in their entirety.

### SUMMARY

Embodiments of the invention are directed to phototherapy devices. The phototherapy device may be capable of being used for the treatment of neonatal jaundice, psoriasis, acne, seasonal affective disorders, and circadian rhythm maintenance disorders, for example. Embodiments of the phototherapy device may comprise organic light emitting diodes. Organic light emitting diodes are advantageous for use in phototherapy devices because they are more cost effective, generate less heat, are more versatile, and provide a wider bandwidth of light than other forms of lighting including conventional light emitting diodes.

Embodiments of the phototherapy device may comprise at least one OLED emitting at least one therapeutic wavelength and a control module. Embodiments of the control module may control at least one organic light emitting diode based upon a temperature, a desired intensity of light emitted, a desired area of illumination, feedback from a medical sensor, activation and deactivation of at least a portion of the OLEDs, the desired spectrum of the emitted light, length of treatment, or combinations thereof.

### BRIEF DESCRIPTION OF FIGURES

FIG. 1 is a perspective view of an embodiment of a phototherapy device comprising an OLED;

FIG. 1A is a cross sectional view of the embodiment of a phototherapy device comprising an OLED in FIG. 1;

FIG. 2 is a perspective view of an embodiment of a phototherapy device comprising a plurality of individually controllable OLEDs;

FIG. 3 is a perspective view of an embodiment of a phototherapy device comprising at least one OLED on a flexible phototherapy device substrate; and

FIG. 4 is a perspective view of an embodiment of a phototherapy device comprising an OLED for use in a bassinet.

FIG. 5 is a perspective view of an embodiment of a phototherapy device in the form of a garment used in conjunction with a sensor measuring a patient parameter and a feedback system to control the output of the phototherapy device.

### DETAILED DESCRIPTION

The invention relates to phototherapy devices comprising at least one OLED. The use of OLEDs in phototherapy devices offers several advantages, for example, OLEDs may provide an increased luminescence over other LEDs or, increase spectral bandwidth, allow for greater intensity control, less heat generation, ease of processability of materials and components during device fabrication, lower weight for portable phototherapy devices, large area devices, uniform illumination, flexible devices, and lower cost for disposable phototherapy treatment device. Embodiments of the phototherapy device may further comprise a phototherapy device substrate that is rigid, conformable, flexible, or a combination thereof allowing production of many varieties of phototherapy devices. OLEDs are generally known in the art as described in, for example, Organic Light-Emitting Materials and Devices, edited by Li and Meng, 2007.

Generally, specific embodiments of the invention include a phototherapy device comprising at least one organic light emitting diode. The device may be in the form of a chamber, garment, pad, mattress, bed sheet, crib, mask, hat, probe, wand, wrap, or an illumination panel that can be free-standing or mounted on a wall or ceiling. The phototherapy device may also comprise a control module to control the organic light emitting diode. The control module may be capable of controlling any parameter of any of the components of the phototherapy device. For example, the control module may control the intensity of the light emitted from the phototherapy device, the temperature of the phototherapy device or of the illuminated object or patient, activation and deactivation of at least a portion of the OLEDs or portions of the OLEDs, the desired spectrum of the emitted light, the length of the treatment, area of illumination, or combinations thereof. The control module may be an automatic control module that adjusts the properties of the phototherapy device based upon input from sensors. Such sensors include, but are not limited to, temperature sensors, light emission spectrum sensors, pressure sensors, light intensity sensors, medical sensors (that measure patient parameters such as by contact or noncontact such as, for example, bilirubin levels in the blood, body temperature, skin temperature, heart rate, oxygen level, carbon monoxide level, carbon dioxide level, bilirubin level, skin color, blood content, bodily fluid content, blood sugar content) as well as other sensors known in the art. Such sensors may be used in combination, for example, to calculate the patient's overall exposure to the phototherapy treatment sensing the intensity of the therapeutic wavelengths over time and the total exposure of all light over time. Also by measuring actual light incident on the patient, the controller may limit exposure to light of certain wavelengths such as ultraviolet light to prevent detrimental effects to the patient because the use of sensors may be used by the control module to calculate and to indicate the total exposure of the patient to light from the phototherapy device and ambient light. Alternatively, the properties of the components of the phototherapy device may be controlled manually. In such embodiments, the phototherapy device may comprise an input device that allows entry of desired control points or manual adjustment of the settings, such as target, maximum, and/or minimum temperature, target spectral output, target, minimum, and/or maximum intensity of the light, target, maximum, and/or minimum time of treatment, target, maximum and/or minimum patient parameters, as well as other parameters. In certain embodiments, the phototherapy device comprises a plurality of OLEDs on a phototherapy device substrate and a control module wherein the various parameters of the output of the OLEDs is controlled by the control module.

The phototherapy device may comprise a single OLED panel, multiple OLEDs in a panel, or multiple organic light emitting diode panels or chips. OLED chips are individually packaged OLED units that can be incorporated in a “plug and play” fashion into a phototherapy device substrate via electrical and/or mechanical couplers. The phototherapy device substrate may include electronics to individually address each chip, or a group of chips coupled to the phototherapy device substrate. Details of OLED chips and their control and configuration can be found in the following United States pending patent applications “OLED CHIP” (Ser. No. 12/543,225 and the corresponding provisional patent application filed Aug. 19, 2008 having Ser. No. 61/090,150); “ORGANIC LIGHT EMITTING DIODE LUMINAIRES” (Ser. No. 12/543,442 and the corresponding provisional patent application filed Oct. 2, 2008 having Ser. No. 61/102,330); and “ORGANIC LIGHT EMITTING DIODE LIGHTING SYSTEMS” (Ser. No. 12/543,440 and the corresponding provisional patent application filed on Oct. 2, 2008 having Ser. No. 61/102,326) which are all hereby incorporated by reference in their entirety. In certain embodiments, the phototherapy device comprises a plurality of OLEDs on a phototherapy device substrate and a control module, wherein the various parameters of the output of the OLEDs is controlled by the control module.

The phototherapy device may further comprise optics to distribute or focus the light. The optics may include, but are not limited to, lenses, waveguides, fiber optics, prisms, diffusive or diffractive optics, as well as other optics. The optics may be capable of distributing or focusing the light to enhance the phototherapy treatment.

In further embodiments, a phototherapy device comprises at least one organic light emitting diode and a heat management system. In an embodiment of the phototherapy device, the heat management system may comprise an air movement device such as a fan. In other embodiments, the heat management system may comprise activating and deactivating OLEDs or adjusting the intensity of the OLEDs to control the temperature. In certain embodiments, the phototherapy device comprises a portion adapted to contact the skin of a patient. The phototherapy device may further comprise an insulation layer between the portion adapted to contact the skin of a patient and the OLED light source. In other embodiments, the phototherapy device may comprise holes to aid in circulation of air against the patient's skin. In embodiments of the phototherapy device, the control module may adjust the heat management system to prevent overheating of the patient, for example, by increasing the speed of a fan or coolant supply.

The phototherapy device described here may comprise a planar light source, or may be of a three dimensional design, for example, formed into an object such as a cylinder or probe. The phototherapy device may be positioned at a distance from the patient, directly in contact with the patient's skin, or it may be positioned internally. For example, the phototherapy device may be positioned in a cavity such as an oral cavity, ear, or the vagina to inhibit bacterial or fungal growth. The phototherapy device may be a large area device used to illuminate the surroundings of a patient, rather than for the illumination of a particular area of tissue on the patient's body. For example, the phototherapy device may be used as a source of light for bright light therapy, wherein the patient is positioned such that their face is angled towards, but not necessarily directly facing, the phototherapy device. The patient may not be directly exposed to the light source, but rather to light reflected by objects illuminated by the phototherapy device.

### Heat Management

Embodiments of the phototherapy device may comprise a heat management system to ensure proper temperature control thereby resulting in safe delivery of treatment to the patient. Excess heat may also reduce the lifetime of an electronic device such as an OLED. Heat management may be desired even though a blue OLED based phototherapy device would produce less heat than halogen or fluorescent lamps because the emission is concentrated in the blue region of the light spectrum. In addition, generally, an OLED device may be more efficient at producing light than conventional lamps. A thermal management system for an embodiment of the phototherapy device can include a separate heat management system for the OLED part of the device and another heat management system for the control module part of the device, such as an air cooling fan system.

A heat management system for the phototherapy device can include a thermally conductive detector module, thermal sensors disposed upon the detector module, and signal processing electronics in thermal communication with the detector module. The thermal detector and sensor system may be used in conjunction with the control module to shut off the phototherapy device, adjust the light intensity, duration, or maintain a programmed intensity cycle to control the temperature of the portion of the phototherapy device in contact with or near the patient. An optically transparent insulation material can also be incorporated between the OLED device and the surface of the phototherapy device which contacts the patient. This could be a thermal gel or pad that acts as a heat sink.

The heat management system may be any configuration that prevents accumulation of heat in any portion of the phototherapy device. In certain embodiments, the phototherapy device comprises a heat sink. A heat sink is a component capable of absorbing and dissipating heat generated in another component. In embodiments of the phototherapy device, the heat sink may be located to draw heat from the treatment portion of the device and dissipate the heat from the non-treatment side of the device. A typical heat sink comprises a metal component with a heat dissipation feature in contact with the source of heat. The heat dissipation feature may be fins, for example. In certain embodiments, the heat management system may comprise a fluid-based cooling system. A fluid-based cooling system includes a base having a first side configured for thermal coupling to the heat generating device and a fluid-based cooling path coupled to the base to provide for heat dissipation through a circulating fluid. In certain embodiments, the heat management device is an electromechanical cooling device such as an active cooling substrate which is a microelectro-mechanical system (MEMS) device which implements the synthetic jet concept into printed wiring board to enhance thermal management. In other embodiments, the heat management system is a thermoelectrical cooling device such as a Peltier cooler.

Embodiments of the phototherapy device may comprise a heat management system that is configured to control the output of at least one organic light emitting diode. Preferably, the heat management system controls a portion of the OLEDs. The heat management system may activate, deactivate, and/or reduce the intensity of the OELDs to control the temperature of the device or the area surrounding the device. The heat management system of the phototherapy device may be capable of activating, deactivating, or reducing the intensity of a portion of OLEDs to manage the localized temperatures or may be capable of activating, deactivating, or reducing the intensity of all the OLEDs. In an embodiment of the phototherapy device comprising OLED chips, the heat management system may be capable of controlling the output of at least one OLED chip. In one embodiment, the heat management system controls the output of the OLED chips by activating and deactivating the OLED chips. In another embodiment, the heat management system controls the output of the organic light emitting diode chips by controlling the intensity of the OLED chips.

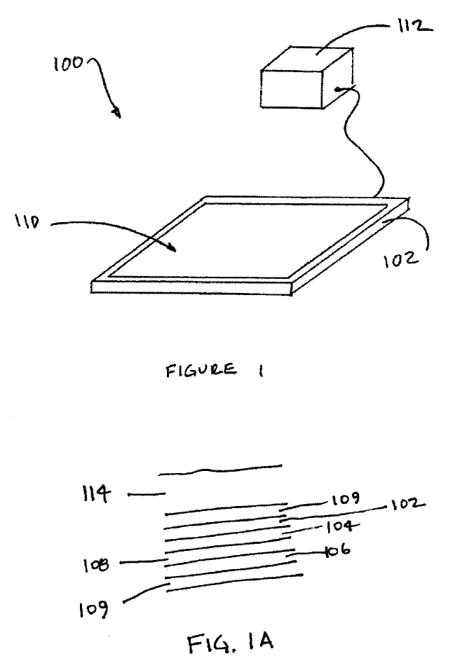

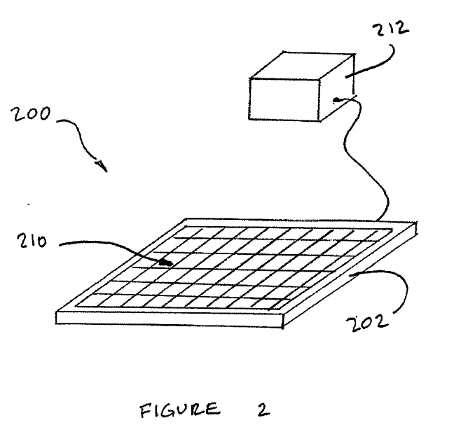

An embodiment of a phototherapy device 100 comprising an OLED is illustrated in FIG. 1 and FIG. 1A. The phototherapy device 100 includes a region comprising a phototherapy lighting region 110 including at least one OLED. Each OLED comprises an OLED device substrate 102, an anode 104, a cathode 106, and an active region 108. The active region 108 comprises an organic material and is electrically coupled to the anode 104 and the cathode 106. The structure of the OLED device may also be an inverted structure wherein the cathode is disposed on the OLED device substrate and the anode is positioned on top of the active region. The embodiment of the phototherapy device 100 comprises a single panel OLED throughout the phototherapy lighting region 110. The phototherapy device may further comprise an encapsulation layer 109 over the light emitting diode components. The individual layers of the OLED are generally described below.

The active region 108 comprises an organic material and is electrically coupled to the anode 104 and the cathode 106. The active region 108 comprises an organic material and, in this embodiment, is configured to emit a broadband emission spectrum with a full width at half maximum (FWHM) greater than about 50 nm. The organic material in the active region may be a polymer, a small molecule, or a combination of a polymer and small molecule with or without a dopant. Using dopants, photons of lower energy/longer wavelength may be generated by higher-energy photons through fluorescent or phosphorescent processes. In addition, an electroluminescent or photoluminescent inorganic light emitter may be incorporated into the OLED device. An example of this type of device is a quantum dot OLED (QD-OLED).

The phototherapy device may also include a light out-coupling component configured to improve light out-coupling. The light out-coupling component may be a lens or array of lenses, a roughened OLED device substrate or interface, a grid or grating, a low refractive index layer, or a photonic crystal. In some embodiments, the light out-coupling layer is also configured to convert the spectrum emitted by the active region to another spectrum of a longer wavelength. For example, the light out-coupling layer may comprise a phosphor layer, or a quantum-dot-based film. The phosphor layer and/or the quantum-dot-based film are configured for down-converting photons of higher energy to lower energy.

The embodiment of the phototherapy device 100 comprises a power supply in housing 112. The power supply is electrically coupled to the anode 104 and the cathode 106. The power supply provides the voltage difference between the anode 104 and the cathode 106 that activates the active region 108 to produce the designed light. The power supply may be connected to a household current, a battery, or other power source. In an embodiment of a phototherapy device 100 for use in the treatment of neonatal jaundice, the active region 108 emits light of a therapeutic spectrum in the blue, blue-green, or green range. The housing 112 may also comprise a control module that is configured to control the light and/or other properties emitted from the phototherapy device 100.

The encapsulation layers 109 isolate the active region 108 from an ambient environment. The encapsulation layers typically comprise a transparent material on the side of the phototherapy device that emits the light. The transparent material may be glass or a plastic, for example. In certain embodiments, the encapsulation 109 prevents water vapor and oxygen to contact and potentially degrade the organic material in the active region 108. In other embodiments, the encapsulation layer 109 may comprise a material that is not completely water vapor and oxygen permeable. The phototherapy device may be further covered with a transparent or semi-transparent covering 114. The covering 114 may provide comfort for a patient using the phototherapy device 100 particularly if the patient is lying on the phototherapy device 100. The covering 114 may provide protection to the phototherapy device, keeping dirt and fluid off of the device and providing a cushion to protect the phototherapy device from impact. The covering may be one of a woven fabric, nonwoven fabric, or plastic film.

The phototherapy device may be used in conjunction with photosensitizing medicines for use in photodynamic therapy treatments. In this case, the phototherapy device activates the photosensitizing medication. The photosensitizing medicine can be a liquid, cream or intravenous drug. For pre-cancer or cancer treatment, for example, the photosensitizing medicine is preferably adsorbed by atypical or cancerous cells. When the photosensitizing medicine is irradiated with light, activated oxygen molecules are produced which can destroy nearby cells.

### Substrates

Further embodiments of the phototherapy device 200 may comprise a plurality of individual OLED panels or chips 210 on a phototherapy device substrate 202. The phototherapy device 200 may further comprise a housing 212 that may comprise a power supply and/or a control module. The control module may comprise a simple on/off control module or may comprise full control over all properties of each individual OLED panel or chip. The plurality of OLED panels or chips 210 may be similar or different. The OLED panels or chips 210 may have different spectral output, different sizes, multiple OLED layers, or other properties that are similar or different than other panels or chips on the phototherapy device 200. In certain embodiments, the at least one organic light emitting diode is in the shape of a disc or a polygon.

An OLED device substrate may be any substance capable of supporting the various layers of the OLED. Many such OLED device substrates are known and described in the art. Descriptions of various OLED device substrates may be found in, for example, Z., Li and H. Meng, Organic Light-Emitting Materials and Devices (Optical Science and Engineering Series). The OLED device substrate may be, for example, rigid, conformable, or flexible. The OLED device substrate can comprise, for example, an inorganic material, an organic material, or a combination of inorganic and organic materials. The OLED device substrate may be, for example, made from metals, glass or plastics. The OLED device substrate may be any shape capable of supporting the other components of the OLED, for example, the OLED device substrate may be substantially flat or planar, curved, or have portions that are substantially flat portions and curved portions. The OLED device substrate may further be, for example, transparent, semi-transparent, translucent, or opaque. A semi-transparent substrate is a substrate that is imperfectly transparent or causes light passing through the substrate to diffuse.

In addition to mechanical support, the OLED device substrate 102 may provide electrical pathways for activating at least one of the OLEDs.

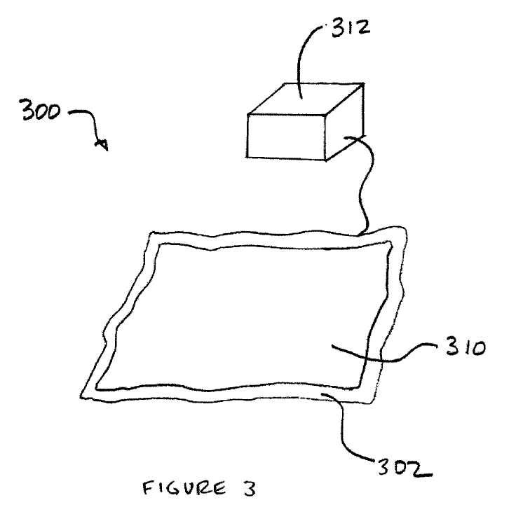

The OLED device substrate may be incorporated into a mattress, pad, bed sheet, hat, robe, pants, shirt, crib sheet, probe, bandage, wrap, or teeth covering strips. The robe may comprise sleeves to provide a comfortable clothing-like phototherapy device. The OLED device substrate may be incorporated into a hand-held wand or a free-standing, wall mounted, or ceiling mounted panel. In certain embodiments, the OLED comprises a single emitter disposed on a flexible phototherapy device substrate. See FIG. 3. Phototherapy device 300 comprises a region comprising a phototherapy lighting region 310 including at least one OLED. The phototherapy device 300 comprises an OLED device. The OLED device comprises an OLED device substrate, an anode, a cathode, and an active region. Similar to the phototherapy device 100, the active region may comprise an organic material and is electrically coupled to the anode and the cathode. The embodiment of the phototherapy device 300 comprises a single panel OLED throughout the phototherapy lighting region 310. The embodiment of the phototherapy device 300 comprises a flexible phototherapy device substrate 302. The flexible phototherapy device substrate 302 allows the phototherapy device to be wrapped around or to cover the patient or a portion of the patient to be treated by the phototherapy.

The embodiment of the phototherapy device 300 comprises a power supply in housing 312. The power supply is electrically coupled to the anode and the cathode. The power supply provides the voltage difference between the anode and the cathode that activates the active region to produce light. The power supply may be connected to household current, a battery or other power source. The power supply may be portable. In an embodiment of a phototherapy device 300 for use in the treatment of neonatal jaundice, the active region emits light in the blue, blue-green, or green region. White light may also be used. The housing 312 may also comprise a control module that is configured to control the light emitted from the phototherapy device 300.

The phototherapy device substrate may be any material capable of attaching to and supporting the emitter. In certain embodiments, the flexible phototherapy device substrate may comprise a plastic film, a woven fabric, a nonwoven fabric, or a cloth. As such, the flexible phototherapy device substrate may comprise natural fiber, synthetic fibers or a combination of natural and synthetic fibers. In alternative embodiments, the OLED may be attached to a rigid phototherapy device substrate. In certain embodiments, the phototherapy device may comprise rigid OLED devices disposed on a flexible phototherapy device substrate. In certain embodiments, the phototherapy device may comprise a transparent cover, covering the rigid OLED device substrates and/or the phototherapy device substrate. The transparent cover may provide comfort to the patient and/or protection to the OLED device and may comprise a woven fabric, nonwoven fabric, or plastic film.

### Anodes

Typically, the anode provides electrical communication between a power source and the active region and, therefore, may comprise any electrically conductive material. Many types of anodes are generally known and described in the art. In certain embodiments, it may be desirable for the anode of the OLED device to be transparent to the light emitted from the active region. The anode may comprise, for example, a transparent conductive oxide (TCO), such as, but not limited to, indium tin oxide (ITO), zinc oxide (ZnO), and the like.

For example, ITO in the form of thin layers (typically, but not necessarily, less than 100 nm thick) is substantially transparent to visible light. The desired thickness of the ITO layer will depend on the application of the device and the desired degree of transparency. In certain embodiments, the anode may have a relatively high work function that helps the injection of holes into the active region. An ITO anode is especially desirable as it may be coated on many different OLED device substrates.

### Cathode

A cathode also provides electrical communication between a power source and the active region. Therefore, a cathode may comprise any electrically conductive material. Many cathodes are generally known and described in the art. A cathode may comprise, for example, a thin metal film such as aluminum or calcium, or a non-metal conductive layer. In certain embodiments, it may be desirable for the cathode of the OLED device to be transparent to the light emitted from the active region. In one embodiment, the anode, cathode, and OLED device substrate are substantially transparent such that the at least one organic light emitting diode is configured to emit light in both the anode and cathode directions. The cathode typically has a relatively low work function to help injection of electrons into the active region. Cathodes may be any thickness, but in typical devices comprising OLEDs, the cathode 106 has a thickness between 100-200 nm.

### Active Region

The active region of the OLED produces the light emitted from the device. In certain embodiments, the active region comprises an organic material, such as an electrically conductive polymer. In the active region, electrons and holes recombine to radiate photons. The radiative photon energy emitted from the active region corresponds to the energy difference between the lowest unoccupied molecular orbital (LUMO) level and the highest occupied molecular orbital (HOMO) level of the organic material. Photons of lower energy/longer wavelength may be generated by higher-energy photons through fluorescent or phosphorescent processes. The active region may comprise at least one of a hole injection layer, hole transfer layer, hole blocking layer, electron injection layer, electron transfer layer, or electron blocking layer. The hole injection layer may comprise one or more polythiophenes, for example.

Polythiophenes can be homopolymers, copolymers, or block copolymers. Synthetic methods, doping, and polymer characterization, including regioregular polythiophenes with side groups, is provided in, for example, U.S. Pat. Nos. 6,602,974 to McCullough et al. and 6,166,172 to McCullough et al., which are hereby incorporated by reference in their entirety. Additional description can be found in the article, “The Chemistry of Conducting Polythiophenes,” by Richard D. McCullough, Adv. Mater. 1998, 10, No. 2, pages 93-116, and references cited therein, which is hereby incorporated by reference in its entirety. Another reference which one skilled in the art can use is the Handbook of Conducting Polymers, 2^{nd} Ed. 1998, Chapter 9, by McCullough et al., “Regioregular, Head-to-Tail Coupled Poly(3-alkylthiophene) and its Derivatives,” pages 225-258, which is hereby incorporated by reference in its entirety. This reference also describes, in chapter 29, “Electroluminescence in Conjugated Polymers” at pages 823-846, which is hereby incorporated by reference in its entirety.

Polythiophenes are described, for example, in Roncali, J., Chem. Rev. 1992, 92, 711; Schopf et al., Polythiophenes: Electrically Conductive Polymers, Springer: Berlin, 1997. See also for example U.S. Pat. Nos. 4,737,557 and 4,909,959.

Block copolymers are described in, for example, Block Copolymers, Overview and Critical Survey, by Noshay and McGrath, Academic Press, 1977. For example, this text describes A-B diblock copolymers (chapter 5), A-B-A triblock copolymers (chapter 6), and (AB)ₙ multiblock copolymers (chapter 7), which can form the basis of block copolymer types in the present invention.

Additional block copolymers including polythiophenes are described in, for example, Francois et al., Synth. Met. 1995, 69, 463-466, which is incorporated by reference in its entirety; Yang et al., Macromolecules 1993, 26, 1188-1190; Widawski et al., Nature (London), vol. 369, Jun. 2, 1994, 387-389; Jenekhe et al., Science, 279, Mar. 20, 1998, 1903-1907;Wang et al., J. Am. Chem. Soc. 2000, 122, 6855-6861; Li et al., Macromolecules 1999, 32, 3034-3044; Hempenius et al., J. Am. Chem. Soc. 1998, 120, 2798-2804;

The following article describes several types of regioregular systems beginning at page 97 and references cited therein: “The Chemistry of Conducting Polythiophenes,” by Richard D. McCullough, Adv. Mater. 1998, 10, No. 2, pages 93-116. In a regioregular polymer, including a polythiophene, the degree of regioregularity can be, for example, about 90% or more, or about 95% or more, or about 98% or more, or about 99% or more. Methods known in the art such as, for example, NMR can be used to measure the degree of regioregularity. Regioregularity can arise in multiple ways. For example, it can arise from polymerization of asymmetric monomers such as a 3-alkylthiophene to provide head-to-tail (HT) poly (3-substituted) thiophene. Alternatively, it can arise from polymerization of monomers which have a plane of symmetry between two portions of monomer such as for example a bi-thiophene, providing for example regioregular HH-TT and TT-HH poly (3-substituted thiophenes).

In particular, substituents which can be used to solubilize conducting polymers with side chains include alkoxy and alkyl including for example C₁ to C₂₅ groups, as well as heteroatom systems which include for example oxygen and nitrogen. In particular, substituents having at least three carbon atoms, or at least five carbon atoms can be used. Mixed substituents can be used. The substituents can be nonpolar, polar or functional organic substitutents. The side group can be called a substituent R which can be for example alkyl, perhaloalkyl, vinyl, acetylenic, alkoxy, aryloxy, vinyloxy, thioalkyl, thioaryl, ketyl, thioketyl, and optionally can be substituted with atoms other than hydrogen.

Thiophene polymers can be star shaped polymers with the number of branches being for example more than three and comprising thiophene units. Thiophene polymers can be dendrimers. See for example Anthopoulos et al., Applied Physics Letters, 82, 26, Jun. 30, 2003, 4824-4826, and further description of dendrimers hereinafter.

Heterocyclic polymers are particularly preferred. A particularly preferred system is the polythiophene system and the regioregular polythiophene system. Polymers can be obtained from Plextronics, Inc., Pittsburgh, Pa. including for example polythiophene-based polymers such as for example Plexcore, Plexcoat, and similar materials.

Another embodiment includes heterocyclic conjugated polymers which are relatively regioirregular. For example, the degree of regioregularity can be about 90% or less, or about 80% or less, or about 70% or less, or about 60% or less, or about 50% or less.

The active region may comprise a continuous region forming a single emitter or a plurality of light emitters. The plurality of light emitters may emit light with substantially different wavelengths. The plurality of light emitters may be vertically stacked within the active region or they may form a mixture. In some embodiments, a dopant is dispersed within an organic host matrix. In one embodiment, a layer of quantum dots is sandwiched between two organic thin films. Alternatively, the plurality of light emitters may comprise a plurality of active regions sharing a common anode and/or cathode. Thus, the plurality of light emitters act as a plurality of light emitting diodes within one OLED device. Stacked OLEDs may be used as well. In this case, individual OLEDs are stacked one on top of another. The stacked configuration generally includes intermediate electrodes disposed between adjacent individual OLEDs such that successive OLEDs share an intermediate electrode and a top electrode of one device is the bottom electrode of another in the stack. The stacked OLEDs may have different active region materials, and therefore, different emissions spectra.

Embodiments of the phototherapy devices comprising at least one OLED may produce light in the visible range (380 to 700 nm), the ultraviolet range (UVA: 315 to 400 nm; UVB: 280 to 315 nm), and/or near infrared light (700 to 1000 nm). Visible light corresponds to a wavelength range of approximately 380 to 700 nanometers (nm) and are usually described as a color range of violet through red. The human eye is not capable of seeing radiation with wavelengths outside this visible spectrum such as in the ultraviolet or infrared range. The visible spectrum from shortest to longest wavelength is generally described as violet (approximately 400 to 450 nm), blue (approximately 450 to 490 nm), green (approximately 490 to 560 nm), yellow (approximately 560 to 590 nm), orange (approximately 590 to 630 nm), and red (approximately 630 to 700 nm). Ultraviolet radiation has a shorter wavelength than the visible violet light and infrared radiation has a longer wavelength than visible red light. White light is a mixture of each of the colors of the visible spectrum.

There are multiple methods of producing white light using OLEDs. One method is to use individual OLEDs that emit visible light in the red range, the green range, and the blue range. The OLEDs may be in a single layer or in a layered structure. Another method comprises preparing an OLED device comprising a phosphor material capable of converting monochromatic light from a blue or UV OLED to broad-spectrum white light or by converting just a portion of the blue light with a yellow emitting phosphor material.

The active region of the OLED device emits a relatively broad band spectrum as compared to inorganic light emitting diodes. The full width at half maximum (FWHM) of the individual spectrum may be larger than 50 nm. In certain applications, such as bright light therapy, the FWHM is preferably larger than about 100 nm, and may be even larger than about 200 nm in some cases. In some cases, the OLED device may produce a narrow band spectrum with a FWHM less than about 50 nm. This may be advantageous in certain phototherapy applications where the tissue or photosensitizing medication responds to a narrow wavelength range. The emission spectrum may be one selected from a NIR, UV, white, a red, a green, a blue, a yellow, an orange, a cyan, or a magenta spectrum or a combination thereof. By appropriately mixing different OLED devices, the output spectrum may be visually substantially white. The broadband spectra of individual OLED devices may be mixed to form an output spectrum which may be very close to naturally white light to human eyes.

Advantageously, OLEDs are currently more efficient in producing green light than inorganic LEDs and less efficient at producing blue light. There is evidence to suggest that blue light may cause retinal damage and promote age-related macular degeneration. Green light is much less damaging to the eyes. Efficient bright light therapy devices can be made with OLED devices that limit or eliminate the amount of damaging blue light emitted.

The active region of the OLED device may be substantially transparent. When mostly transparent layers are used, a plurality of OLED devices may be vertically stacked without substantially blocking light emission from individual devices. In addition, an OLED chip may include a plurality of vertically-stacked transparent OLEDs, which are not stand-alone devices as they may not have their own encapsulations, but may have their own OLED device substrates and electrodes and can be configured to be individually controlled.

The active region may comprise a single or multiple layers, for example, a combination of p- and n-type layers. The p- and n-type materials may be bonded to each other in the active region. The bonding may be ionic or covalent bonding, for example. The multiple layers of the active region may form heterostructures therebetween.

The active region may be manufactured by known methods including, for example, spin casting, drop casting, vapor deposition or sputtering, crystalline growth, patterned etching, dip coating, or by printing techniques such as ink jet printing, off-setting, transfer processes, or by spray applications.

The ability to fabricate OLEDs using solution deposition techniques allows for low-cost, large-area devices. This is an advantage for phototherapy devices where the treatment area is a large area. Also, large area devices may have fewer thermal issues than point source light devices. The active region may have an emissive area of any size. In one embodiment, the emissive area of individual OLED devices is about 1 cm². In another embodiment, the emissive area is less than 1 m². In another embodiment, the emissive area is larger than about 0.1 cm².

### Organic Material of the Active Region

The active region may comprise an organic material. The organic material in the active region 108 may comprise an electroluminescent material, such as an electroluminescent polymer. An electroluminescent material emits light in response to an electrical stimulation such as an electric current or to a strong electric field.

An electroluminescent polymer may be a fluorescent emitter or a phosphorescent emitter. Electroluminescent polymers include, but are not limited to, poly-phenylene vinylene, or polyfluorene, for example. The polymers are often engineered to substitute side chains onto the backbone of the polymer chain to tune the color emitted from the active region, improve the solubility and stability of the active region, or to improve the ease processing of the polymers into an OLED. Alternatively or in combination, small molecule emitters may also be used in the OLED. Small molecule emitters include, but are not limited to, organo-metallic chelates or conjugated dendrimers, for example.

### Electrical Coupling

In certain embodiments, the OLED relies on electrical communication from the anode through the active region to the cathode. The electrical coupling between the active region and the anode or cathode may be made by direct contact between the components or may comprise additional layers as discussed in detail above.

### Power Supply

The power supply may be any power supply capable of supplying sufficient power to activate the active region. The power supply may comprise a battery, solar cell, fuel cell, an adapter, or may be part of a power grid. The OLED devices may be powered by AC or DC current.

### Control Module

The control module may comprise an input means allowing selection of the treatment to be provided, the treatment time, the intensity of the treatment, the spectral output of the phototherapy device, and/or the age of the patient. The input means may include, but are not limited to, a keyboard, keypad, mouse, touch screen, buttons, or other input devices. The control module may additionally have a screen that provides information concerning the treatment, the temperature of one or more locations of the phototherapy device or the patient, exposure area of patient, the length of the treatment cycle, the time remaining in the treatment cycle, light intensity setting of the OLED, and if more than one independently controlled OLED, the light intensity of a plurality of OLEDs, the cumulative time of multiple treatments by the phototherapy device, and other information concerning the settings and operation of the phototherapy device. The control module may vary at least one of a voltage, current, a pulse width, or a pulse frequency to control at least one organic light emitting diode, for example. The screen may also provide information concerning preprogrammed treatment cycles for various phototherapy treatments.

The control module may independently regulate each of the OLEDs by adjusting the activation and deactivation, the degree of activation, spectral emissions, and intensity of the independently regulated OLED devices. The control module may operate based upon preprogrammed treatment cycles or allow dynamic control of the treatment cycle based upon user input or input from various sensors connected to control module of the phototherapy device. For example, the phototherapy device may comprise independent temperature sensors for sensing the temperature of at least a portion of the phototherapy device. The temperature sensors may preferably be in a portion of the phototherapy device that may be in contact with the patient or the patient's clothing. In other embodiments, the temperature sensors may read the temperature of the patient by direct contact with the patient or without direct contact such as with an infrared temperature sensor.

A sensor providing input to the control module may comprise various types of signals, for example, electrical, mechanical, or pneumatic, that corresponds to the parameter being sensed. Such inputs could include patient parameters, as previously defined. Furthermore, sensors can be used to measure degradation of one or more OLEDs in the phototherapy device. The sensor may be a light sensor such as an intensity meter or spectrometer. The various inputs from the sensors allow the control module to determine and/or indicate the degree of phototherapy treatment delivered to the patient during single or multiple treatments. In such embodiments, the control module is capable of being programmed for treatment of neonatal jaundice, acne, psoriasis, seasonal affective disorders, circadian rhythm maintenance disorders, and/or other diseases, syndromes, conditions, or illnesses that are capable of being treated by phototherapy. In addition, feedback from the sensors may allow the control module to adjust the driving conditions of the one or more OLED devices in the phototherapy device to compensate for example, for OLED device degradation or spectral shift and to help maintain the desired light output parameters. Control and feedback systems are described in more detail in United States pending patent applications “OLED CHIP” (Ser. No. 12/543,225 and the corresponding provisional patent application filed Aug. 19, 2008 having Ser. No. 61/090,150); “ORGANIC LIGHT EMITTING DIODE LUMINAIRES” (Ser. No. 12/543,442 and the corresponding provisional patent application filed Oct. 2, 2008 having Ser. No. 61/102,330); and “ORGANIC LIGHT EMITTING DIODE LIGHTING SYSTEMS” (Ser. No. 12/543,440 and the corresponding provisional patent application filed on Oct. 2, 2008 having Ser. No. 61/102,326) which are all hereby incorporated by reference in their entirety.

The phototherapy treatment may be divided into a number of treatment sessions that added together result in an overall treatment time. For such cases, the control module may comprise at least one timer configured to measure session time and overall treatment time or both. The timer may be used simply to monitor the session time or overall treatment time or may be used to deactivate the phototherapy device after completion of a session or overall treatment. The control module may also comprise a determination of the accumulated degree of treatment. The degree of phototherapy treatment may be calculated from the total area of OLED that are activated, the overall treatment time, the intensity of the light emitted by the OLED to the patient, and/or the spectral output of the phototherapy device compared to the type of treatment being provided.

For example, if the temperature of the phototherapy device is considered to be too high and the intensity of the OLED is reduced to lower the temperature, the control module may increase the time of the treatment to compensate for the lower intensity and provide the overall degree of the desired treatment. A summation function may be used to calculate an accumulated treatment factor for each light component. All OLED components accumulated exposure quotients are tallied to determine the total degree of treatment.

The phototherapy device may comprise the control module. The control module may include a processor and memory. In an embodiment wherein the OLED device includes individually controllable OLEDs or separate OLED devices, each of the individual OLEDs or devices may be assigned a logical address and controlled by the control circuit or software in the control module through one of their logical addresses. The control module may individually address and control the OLED devices to adjust the color, pattern, or brightness of the OLED.

The control module may adjust the color of the output of the OLED device by selectively driving at least some of the plurality of OLED devices differently from other OLED devices. Selectively driving some of the plurality of OLED devices differently from other OLED devices may be realized by, for example, selectively varying a drive voltage or a drive current of the OLED devices. The plurality of OLED devices can be controlled by the control module by any means such as, but not limited to, through digital-to-analog converters (DAC), respectively. The OLED devices can have different emission spectra, such as red, green, and blue, NIR or UV. The DAC can deliver drive current pulses of suitable amplitudes and widths to their respective OLED devices. The OLED devices may be driven independently, collectively, or interdependently.

The control module can further comprise an input/output (I/O) interface to receive the feedback data from the sensors. Memory can be included in the control module to store commands to generate drive sequences. A clock can be used to synchronize the drive sequences. The control module can further comprise a data port to receive command data, and the command data can come from a user, a processor, or a computer. The control module can further comprise other components generally known in the art, such as shift registers.

A pressure switch may be incorporated into an embodiment of the phototherapy device that is intended for use by placing a patient on top of the device. The control module may automatically deactivate the device if the patient is removed from the phototherapy device. Such an embodiment may be used for treatment of neonatal jaundice. When a baby is placed on the phototherapy device the device may automatically activate and when the baby is removed from the phototherapy device after treatment, the phototherapy device is automatically deactivated.

The control module can be implemented using, for example, a computer with suitable control software and additional discrete components, or using an application specific integrated circuit (ASIC).

### Encapsulation

The OLED device may be already packaged in an encapsulation that protects the organic material of the OLED device from the ambient environment. The resulting OLED device may thus be a standalone device that can be readily installed in a system which does not necessarily provide oxygen and water vapor barriers.

An encapsulation may comprise a housing forming an enclosure around the active region. The encapsulation may comprise a housing sealed to the OLED device substrate. A sealant may additionally be disposed between the housing and the OLED device substrate and may form an oxygen and water vapor barrier for the active region. The housing may have an electrically conductive path disposed through an OLED device substrate or encapsulation. The electrically conductive paths may be electrically coupled to the cathode or the anode.

In disposable or single use embodiments of the phototherapy device, the encapsulation may comprise an encapsulation layer that allows some permeability to oxygen and water vapor. OLED devices are encapsulated to prevent or limit the amount of water and moisture that may come in contact with the active region. The performance of devices like organic light emitting diodes (OLEDs) and solar cells is sensitive to moisture because water and oxygen molecules seep past the protective plastic layer over time and degrade the organic materials which form the core of these products.

In certain embodiments, layers used to protect these materials have a water vapor transmission rate of less than 10⁻² g/m² per day at 25° C. and 90% relative humidity. OLED device manufacturers are attempting to produce encapsulation layers with much lower water vapor transmission rates. However, defects such as pinholes, cracks and grain boundaries are common in barrier films fabricated onto plastic substrates. Oxygen and water molecules are able to seep through and penetrate the plastic barrier through such defects.

Currently, OLED device manufacturers apply multiple barrier layers to reduce the effect of such defects, for example some OLED devices may comprise both organic and inorganic barrier layers. These multiple layers create a tortuous lengthy pathway for water and oxygen providing a more effective barrier. Embodiments of the phototherapy device comprise at least one OLED and an encapsulation having a water vapor transmission rate of less than 10⁻² g/m² per day at 25° C. and 90% relative humidity.

However, such low water or oxygen transmission rates may not be necessary for disposable or single use phototherapy devices. Therefore, the same degree of encapsulation is not necessary for an OLED device that is designed to last for less than 100 hours of use as is required for an OLED device that is designed to last for greater than 1000 hours of use. Embodiments of the phototherapy device comprise one or more barrier layers that together provide a water vapor transmission rate such that the OLED device is functional as a phototherapy device for less than 100 hours, such as a single use neonatal jaundice phototherapy device. In other applications, the phototherapy device may comprise one or more barrier layers that together provide a water vapor transmission rate such that the OLED device is functional as a phototherapy device for less than 50 hours or even 24 hours, such as a daily use acne phototherapy device or a phototherapy device to use with photosensitive medications. Other embodiments of the phototherapy device may comprise one or more barrier layers that together provide a water vapor transmission rate such that the OLED device is functional as a phototherapy device for less than 10 hours. The single use or disposable OLED phototherapy devices may comprise less expensive materials and simpler processing to produce. Typically, more permeable materials are also less expensive and easier to process than the materials required to produce less permeable barrier layers.

In a further example, a single use phototherapy device may be used to treat a single case of neonatal jaundice at home, for example. The phototherapy device may be configured to provide 100 hours of phototherapy or other prescribed length of time. Such an embodiment may be prescribed by a physician for home use by a new parent. The parent of the neonate patient could be confident that they are providing the prescribed treatment successfully and the phototherapy device may be inexpensively used and produced. The control module may be preprogrammed to provide a certain number of timed treatment sessions at a specified light intensity. This provides the ability to the infant and parent to return home to continue the treatment for neonatal jaundice rather than remain in the hospital to receive more expensive treatments and expensive hospital room charges. The disposable nature of the device also avoids hassles associated with replacement or return of leased or rented home-treatment equipment.

### Phototherapy

The phototherapy device is capable of being used to treat any disease, syndrome, disorder, condition, or illness that responds to phototherapy such as, but not limited to, neonatal jaundice, acne, psoriasis, eczema, cancer, pre-cancer, actinic keratosis, thyroid disorders, sleep disorders, neuropathy, seasonal affective disorders, depression, bulimia, inflammation, arthritis, Reynaud's syndrome, poor circulation, irritable bowel syndrome, obesity, tremors associated with Parkinson's disease, ulcers, infections, and circadian rhythm maintenance disorders, for example. The phototherapy device may be used to promote relaxation, wound-healing, enhance fertility, stimulate hair growth, promote weight loss, and decrease the appearance of cellulite and aging of the skin. The phototherapy device may be used as an alternate to acupuncture for trigger point therapy. Phototherapy can be provided in isolation or in combination with a photoactive topical medication or drug that can be applied over the treatment area on the body of the patient.

For certain treatments, embodiments of the phototherapy device may comprise an enclosure wherein the entire patient or just the portion to be treated is exposed to light of specific wavelengths. An enclosure such as a light box or a booth can be used for this purpose. An overhead spot light source can be provided. Alternatively, portable or hand-held phototherapy devices can also be used to provide treatment over a smaller area of the patient's body as in the case of acne treatment. The advantage of a portable phototherapy system is that treatment can be achieved at home and as per the patient's convenience instead of in the physician's clinic or hospital. Infants can be treated for neonatal jaundice using a portable phototherapy system at home, for example. In addition to being much cheaper to accomplish, this form of therapy is much more conducive to maintaining normal mother-infant interactions.

The phototherapy device may be a single use phototherapy device. A single use phototherapy device may be used to treat a single case of neonatal jaundice or provide a daily treatment of acne through a disposable light patch, for example.

### Neonatal Jaundice

In the case of neonatal jaundice, babies may be born with a high level of bilirubin in their blood. Bilirubin is a fat soluble compound and is therefore not easily removed from the body by natural processes. It has been found that exposure to light, specifically light in the blue, blue-green, and/or green spectrum, results in the conversion of the toxic bilirubin into a structural isomer, lumirubin. Lumirubin is water soluble and may more easily be expelled by the body. Exposure of the infant to blue to green light results in decrease in the levels of bilirubin thereby achieving treatment of neonatal jaundice. The phototherapy for the treatment of jaundice should include light in the blue to green band in the range of 410 nm to 550 nm.

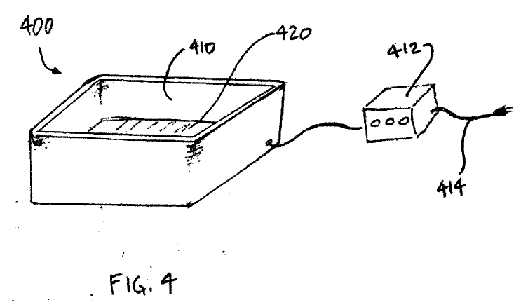

There are various embodiments of a phototherapy device for treatment of neonatal jaundice. For example, embodiments of a phototherapy device for treatment of a neonatal jaundice include overhead spot lights, flexible phototherapy device substrates, comprising OLED such as blankets comprising OLEDs and/or pads comprising OLEDs, rigid phototherapy device substrates comprising OLEDs such as panels or bassinets comprising OLEDs as well as other configurations. One embodiment is shown in FIG. 4. The embodiment of FIG. 4 comprises a bassinet 400 comprising at least one OLED 410. The OLED may line the inside surface of the sides of the bassinet 400 and/or the inside surface of the bottom of the bassinet 400. If the inside surface of the bottom of the bassinet 400 comprises OLEDs, the bassinet 400 may comprise a transparent pad 420 to provide comfort to a patient.

Embodiments of the bassinet 400 may further comprise a control module 412 having functionality as described herein and a power supply such as, but not limited to, a power cord 414 for connecting to household current.

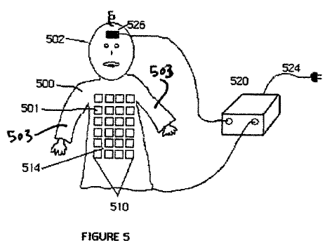

A further embodiment of a phototherapy device for treatment of neonatal jaundice is shown in FIG. 5. The embodiment of the phototherapy device of FIG. 5 comprises a garment 500 comprising sleeves 503 and at least one OLED 501. In this embodiment, the garment serves as the phototherapy device substrate and is flexible. The garment may be made of a fabric material for patient 502 comfort. The OLED devices 501 are disposed on the garment phototherapy device substrate. The OLEDs may form an array 510 on the interior and exterior front and/or back of the garment. The array may be a closely packed array, or the OLED devices may have some space 514 between them as shown in FIG. 5. The space 514 between the OLEDs may serve as positions for sensors integrated into the device and may comprise the circuitry to power the individual OLEDs in the array. The space 514 between the OLED devices may provide room for holes to be placed in the garment for thermal management. Holes may also provide access to the patient's skin and room for sensor leads, for instance

The garment 500 may be lined with a transparent phototherapy device cover to provide comfort to the patient 502. Embodiments of the garment 500 may further comprise a control module 520 having a functionality described herein and a power supply such as, but not limited to, a battery or a power cord 524 for connecting to household current. The garment phototherapy system may further comprise a sensor 526 to measure a patient parameter such as serum bilirubin values. The sensor 526 may be a transcutaneous bilirubinometer. The control module 520 may program the sensor 526 to take a patient parameter measurement at scheduled intervals. The value of the measurement may be used by the control module to adjust the spectrum, intensity, or activation and deactivation of OLED devices 501 in the garment 500. Temperature sensors may be incorporated into the garment 500. The temperature sensors may be incorporated into the garment and positioned such that are new or in contact with the patient's skin. There can be more than one temperature sensor integrated into the device at various positions. For instance, they can be positioned to measure temperatures on the back, chest, and stomach. Again, the control module 520 may schedule the timing of temperature measurements and may use the values to control the output of the device. The control module 520 may be used in conjunction with several types of sensors in the same phototherapy device.

### Seasonal Affective Disorder

Seasonal affective disorders are a mild disorder in which people with typically normal mental health experience a mild depression in a certain season, usually winter. While full sunlight may be preferred for treatment of certain seasonal affective disorders, phototherapy has been effective for the treatment of seasonal affective disorder when consistent exposure to full sunlight is not available to or inconvenient for the patient. Though traditionally treated by white light, light in the blue spectrum has been found to be at least as efficacious as white light. For older people, blue light may be no more effective than light in the red or green ranges. Generally, the most effective wavelengths of blue light for treatment of seasonal affective disorder are in the range of 460 nm to 485 nm. Embodiments of a phototherapy device for treatment of seasonal affective disorder include overhead spot lights, light boxes or booths, flexible phototherapy device substrates, comprising OLED such as blankets comprising OLEDs and/or pads comprising OLEDs, rigid phototherapy device substrates comprising OLEDs such as panels comprising OLEDs that can be mounted in the home, office, as well as other configurations.

### Acne

Methods of treating acne with phototherapy may comprise light in the visible violet region in the range 405-420 nm. Such exposure may activate a porphyrin (Coproporphyrin III) in Propionibacterium acnes which damage and ultimately kill the bacteria. A total of 320 J/cm² of light within this range may be sufficient to kill the bacteria. Such visible violet light does not comprise light in the ultraviolet range and, therefore, produces little chance of tanning or sunburn through use of the phototherapy device. Treatment is often accompanied by application of red light which has been shown to activate ATP in human skin cells and improve response rates.

Embodiments of a phototherapy device for treatment of acne include overhead spot lights, light boxes or booths, flexible phototherapy device substrates, comprising OLED such as blankets comprising OLEDs and/or pads comprising OLEDs, rigid phototherapy device substrates comprising OLEDs that can be incorporated into hand-held devices as well as other configurations.

### Sterilization

A phototherapy device comprising UV OLED may be used to sterilize or disinfect surfaces during or between phototherapy treatments. Embodiments of the phototherapy device may comprise OLEDs capable of emitting light in the treatment range and at least one OLED emitting light in the UV range to maintain sterile surfaces exposed to the light. The intensity of the UV OLED must be kept low during treatment cycles, however, to avoid skin damage to the patient. A sterilization cycle incorporating higher intensity UV light may be used between treatment cycles to sterilize the phototherapy device and anything sufficiently exposed to the light emitted from the phototherapy device.

Embodiments include methods of treating a patient comprising diagnosing a patient with a condition that may be treated with phototherapy, subjecting the patient to phototherapy wherein the phototherapy is delivered by a phototherapy device comprising an at least one OLED and a control module. The control module may include sensors, as previously described, to control or monitor the phototherapy treatment.

The phototherapy treatment may be applied based upon the specific condition of the patient. The light output of the phototherapy device may be substantially limited to the therapeutic wavelengths for the specific condition or the output of the phototherapy device may include the therapeutic wavelengths of the condition. Therapeutic wavelengths include any wavelengths that provide a therapeutic benefit to the patient generally and, more particularly, provide therapeutic benefit to the patient for a specific condition for which the treatment is prescribed. The therapeutic wavelengths may all fall within one range or may include more than one range of wavelengths. For example, if the condition to be treated is hyperbilirubinemia, the therapeutic wavelength may be considered to be in the range of 410 to 550 nm

Embodiments of the method may comprise determining at least one therapeutic wavelength to treat said condition and exposing a patient to the light source emitting said at least one therapeutic wavelength. The method may further comprise exposing an internal area of the body to the therapeutic wavelengths.

Further methods of treating a patient comprising diagnosing a patient with a condition that may be treated with phototherapy may comprise administering a photosensitive mediation to the patient. The photosensitive medication may be administered by any means of administering a pharmaceutical including orally, intravenously, parenterally, topically, or other means.

The methods may include a feedback control module wherein the control module controls and monitors the phototherapy. The control module may be capable of determining the overall intensity of the phototherapy treatment, limit the exposure of the patient to harmful exposures, adjust the treatment based upon the response of the patient such as the patient's internal or skin temperature, heat rate, or skin color, for example.

While the invention has been disclosed in its preferred forms, it will be apparent to those skilled in the art that many modifications, additions, and deletions can be made therein without departing from the spirit and scope of the invention and its equivalents as set forth in the following claims.

1. A phototherapy device, comprising:
at least one organic light emitting diode emitting at least one therapeutic wavelength; and
a control module to control at least one organic light emitting diode based upon a temperature, a desired intensity of light emitted, activation and deactivation of at least a portion of the organic light emitting diodes, input from a sensor, the desired spectrum of the emitted light, the length of the treatment, area of illumination or combinations thereof.

2. The phototherapy device of claim 1, wherein the phototherapy device is capable of being used for the treatment of one or more medical conditions selected from the group consisting of acne, psoriasis, eczema, cancer, pre-cancer, depression, bulimia, actinic keratosis, thyroid disorders, seasonal affective disorders, and circadian rhythm maintenance disorders, neuropathy, wrinkles, cellulite, sleep disorders, tremors associated with Parkinson's disease, poor hair growth, poor fertility, obesity, wounds, poor circulation, irritable bowel syndrome, colic, inflammation, arthritis, Reynaud's syndrome, and infections.

3. The phototherapy device of claim 1, wherein the phototherapy device is capable of treating neonatal jaundice.

4. The phototherapy device of claim 3, wherein the OLED is capable of emitting a wavelength for activating a pharmaceutical compound.

5. The phototherapy device of claim 4, further comprising the pharmaceutical compound to be activated by said organic light emitting diode for photodynamic therapy treatment.

6. The phototherapy device of claim 1, wherein the phototherapy device is capable of being used for teeth whitening, weight loss, hair growth, wrinkle reduction, evening skin tone, or for reducing the appearance of cellulite.

7. The phototherapy device of claim 1, wherein the organic light emitting diode comprises an OLED device substrate; an anode; a cathode; an active region comprising an organic material, wherein the active region is electrically coupled to the anode and the cathode; and an encapsulation over the organic light emitting diode.

8. The phototherapy device of claim 7, wherein at least one of the anode and cathode is transparent.

9. The phototherapy device of claim 7, wherein the anode, cathode, and OLED device substrate are substantially transparent such that the at least one organic light emitting diode is configured to emit light in both the anode and cathode directions.

10. The phototherapy device of claim 7, wherein the active region is configured to emit a broadband emission spectrum with a full width at half maximum larger than about 50 nm.

11. The phototherapy device of claim 7, wherein the active region is a continuous region forming a single emitter.

12. The phototherapy device of claim 7, wherein the active region comprises a plurality of light emitters.

13. The phototherapy device of claim 12, wherein the control module controls activation, deactivation, and activation levels of at least a portion of the light emitters.

14. The phototherapy device of claim 12, wherein the control module controls the level of intensity of the light emitters.

15. The phototherapy device of claim 7, wherein the active region further comprises an inorganic light emitter.

16. The phototherapy device of claim 15, wherein the inorganic light emitter is quantum dots or an inorganic phosphorescent material.

17. The phototherapy device of claim 7, wherein the active region comprises at least one of a polymer emitter or a small molecule emitter.

18. The phototherapy device of claim 7 or 12, wherein the active region comprises a plurality of vertically stacked light emitting layers.

19. The phototherapy device of claim 18, wherein at least two of the plurality of vertically stacked light emitting layers are configured to emit light of different colors.

20. The phototherapy device of claim 7, wherein the active region comprises at least one of a fluorescent emitter or a phosphorescent emitter.

21. The phototherapy device of claim 7, wherein the organic light emitting diode further comprises at least one hole injection layer.

22. The phototherapy device of claim 21, wherein at least one hole injection layer comprise at least one polythiophene.

23. The phototherapy device of claim 22, wherein at least one hole injection comprises at least one planarizing agent.

24. The phototherapy device of claim 7, wherein the organic light emitting diode further comprises at least one electron transport layer.

25. The phototherapy device of claim 7, further comprising one or more of a hole blocking layer, electron blocking layer, hole transport layer, electron transport layer, hole injection layer, or electron injection layer.

26. The phototherapy device of claim 7, wherein the encapsulation comprises a water and air barrier material with oxygen and water vapor transmission rates such that the phototherapy device is capable of performing for less than 100 hours of therapeutic use.

27. The phototherapy device of claim 7, wherein the encapsulation comprises a water and air barrier material with oxygen and water vapor transmission rates such that the phototherapy device is capable of performing for less than 1000 hours of therapeutic use.

28. The phototherapy device of claim 7, wherein the encapsulation comprises a water and air barrier material with oxygen and water vapor transmission rates such that the phototherapy device is capable of performing for a single therapeutic use.

29. The phototherapy device of claim 7, wherein the active region has an emissive area greater than 0.1 cm².

30. The phototherapy device of claim 7, wherein the active region has an emissive area less than 1 m².

31. The phototherapy device of claim 7, wherein the active region has an emissive area of greater than about 0.1 cm².

32. The phototherapy device of claim 1, wherein the at least one organic light emitting diode is a quantum dot organic light emitting diode.

33. The phototherapy device of claim 1, further comprising a transparent or semitransparent covering.

34. The phototherapy device of claim 33, wherein the transparent or semitransparent covering is one of a woven fabric, nonwoven fabric, or plastic film.

35. The phototherapy device of claim 1, wherein the phototherapy device is a pad, chamber, garment, bandage, mask, wrap, bassinet, body covering, probe, wand, or a handheld, freestanding, hanging, or mounted illumination source.

36. The phototherapy device of claim 1, wherein the at least one organic light emitting diode is positioned to form a non-planar emitting surface.

37. The phototherapy device of claim 36, wherein the non-planar emitting surface is cylindrical.

38. The phototherapy device of claim 1, wherein at least a portion of the at least one organic light emitting diodes are vertically stacked.

39. The phototherapy device of claim 1, wherein at least a portion of the organic light emitting diodes emit a therapeutic spectrum for the treatment of hyperbilirubinemia.

40. The phototherapy device of claim 39, wherein the spectrum comprises wavelengths substantially in the range of 410 nm to 550 nm.

41. The phototherapy device of claim 39, wherein the OLEDs are incorporated into a mattress, pad, bed sheet, robe, pants, shirt, torso wrap, or crib sheet, crib siding, crib cover, or bassinet.

42. The phototherapy device of claim 41, wherein the robe or shirt comprises sleeves.

43. The phototherapy device of claim 1, wherein the at least one organic light emitting diode emits light in the near infrared spectrum.

44. The phototherapy device of claim 1, wherein the at least one organic light emitting diode emits light in the visible spectrum.

45. The phototherapy device of claim 1, wherein the at least one organic light emitting diode emits light in the ultraviolet spectrum.

46. The phototherapy device of claim 1, wherein at least one organic light emitting diode is a chip.

47. The phototherapy device of claim 46, wherein one or more organic light emitting diode chips are disposed on a flexible phototherapy device substrate.

48. The phototherapy device of claim 47, wherein the flexible phototherapy device substrate is a woven fabric or cloth.

49. The phototherapy device of claim 48, wherein the flexible phototherapy device substrate comprises natural fiber, synthetic fibers or a combination of natural and synthetic fibers.

50. The phototherapy device of claim 47, wherein the flexible phototherapy device substrate is a plastic film.

51. The phototherapy device of claim 47, wherein the flexible phototherapy device substrate is a nonwoven fabric.

52. The phototherapy device of claim 46, wherein the one or more organic light emitting diode chips are disposed on a phototherapy device substrate that is a rigid substrate, a conformable substrate, a flexible substrate, or combinations thereof.

53. The phototherapy device of claim 1, wherein the at least one organic light emitting diode is in the shape of a disc or a polygon.

54. The phototherapy device of claim 1, wherein the at least one organic light emitting diodes form an array of organic light emitting diodes disposed on a a phototherapy device substrate that is a rigid substrate, a conformable substrate, a flexible substrate, or combinations thereof.

55. A device of claim 54, wherein the OLED device substrate is one of a rigid substrate, a conformable substrate, or flexible substrate.

56. The phototherapy device of claim 1, further comprising a power supply.

57. The phototherapy device of claim 56, wherein the power supply is portable.

58. The phototherapy device of claim 1, further comprising a portion adapted to contact the skin of a patient.

59. The phototherapy device of claim 58, further comprising a heat management system comprising an insulation layer between the portion adapted to contact the skin of a patient and at least one organic light emitting diode.

60. The phototherapy device of claim 1, further comprising holes to provide ventilation.

61. The phototherapy device of claim 1, further comprising a heat management system.

62. The phototherapy device of claim 61, wherein the heat management system comprises an air movement device.

63. The phototherapy device of claim 62, wherein the air movement device is a fan.

64. The phototherapy device of claim 61, further comprising a temperature sensor.

65. The phototherapy device of claim 61, wherein at least one organic light emitting diode is a chip and wherein the heat management system controls the output of at least one organic light emitting diode chip.

66. The phototherapy device of claim 64, wherein the temperature sensor senses a temperature on or near the skin of the patient, an illuminated surface, the at least one organic light emitting diode, or a power supply.

67. The phototherapy device of claim 65, wherein the heat management system controls the output of the organic light emitting diode chips by activating and deactivating the organic light emitting diode chips.

68. The phototherapy device of claim 65, wherein the heat management system controls the output of the organic light emitting diode chips by controlling the intensity output of the organic light emitting diode chips.

69. The phototherapy device of claim 61, wherein the heat management system comprises a heat sink.

70. The phototherapy device of claim 61, wherein the heat management system is a fluid-based cooling system.

71. The phototherapy device of claim 61, wherein the heat management system comprises a thermoelectrical cooling system.

72. The phototherapy device of claim 61, wherein the heat management system comprises an electro-mechanical cooling system.

73. The phototherapy device of claim 1, further comprising optics to distribute or focus light.

74. The phototherapy device of claim 1, wherein the control module is configured to selectively drive at least some of the organic light emitting diode devices to different activation levels to compensate for degradation of some of the organic light emitting diode devices thereby maintaining the desired output color characteristics.

75. The phototherapy device of claim 1, wherein the control module is further configured to tune a color of light emitted from the phototherapy device by selectively driving at least one of the organic light emitting diodes at activation levels different from other organic light emitting diodes.

76. The phototherapy device of claim 1, wherein the control module controls at least one of the intensity, spectrum output by the organic light emitting diodes, and the number of light emitting diodes that are activated.

77. The phototherapy device of claim 1, wherein the light emitting diodes are at least one light emitting diode selected from red organic light emitting diodes, green organic light emitting diodes, blue organic light emitting diodes, yellow organic light emitting diodes, white organic light emitting diodes, and combinations thereof.

78. The phototherapy device of claim 77, wherein the control module controls the number of each color of organic light emitting diodes that are activated and the intensity of the organic light emitting diodes.

79. The phototherapy device of claim 1, wherein the control module varies at least one of a voltage, current, a pulse width, or a pulse frequency to control at least one organic light emitting diode.

80. The phototherapy device of claim 1, further comprising at least one sensor to provide feedback to the control module.

81. The phototherapy device of claim 80, wherein at least one sensor detects a temperature, oxygen level, carbon monoxide level, carbon dioxide level, bilirubin level, skin color, blood content, bodily fluid content, blood sugar content, light intensity, or spectral characteristic.

82. The phototherapy device of claim 1, further comprising a timer.

83. The phototherapy device of claim 82, wherein the timer measures a session time, a total treatment time, or both.

84. A method of carrying out phototherapy, comprising:
diagnosing a condition that can be treated with phototherapy,
determining at least one area of the body to be exposed to phototherapeutic light,
determining at least one therapeutic wavelength to treat said condition, and
irradiating said at least one area of the body with said at least one therapeutic wavelength, said at least one therapeutic wavelength being produced by the phototherapy device of claim 1.

85. A method of carrying out phototherapy, comprising:
diagnosing a condition that can be treated by stimulation of the retina with a therapeutic spectrum,
determining the treatment protocol,
determining at least one therapeutic wavelength to treat said condition, and
positioning a light source emitting said at least one therapeutic wavelength near a patient such that said at least one therapeutic wavelength is visible with a therapeutic intensity by the patient, said light source comprising the phototherapy device of claim 1.

86. The method of carrying out phototherapy of claim 84 or 85, wherein the area of the body to be exposed to phototherapeutic light is an internal portion of the body.

87. The method of carrying out phototherapy of any of claims 84, 85, and 86, further comprising the application of photosensitive medication to the area of the body to be exposed to phototherapeutic light 86. The method of carrying out phototherapy of claim 84, wherein the condition to be treated is hyperbilirubinemia, and the at least one therapeutic wavelength is in the range of 410 to 550 nm.

88. The phototherapy device of claim 6, further comprising a light out coupling component.

89. The phototherapy device of claim 88, wherein the light out coupling component comprises a lens, an array of lenses, a roughened OLED device substrate or interface, a grid, a grating, a low refractive index layer, or a photonic crystal.
